(19) United States
(12) Patent Application Publication
(10) Pub. No.: US 2009/0170195 A1
(43) Pub. Date: Jul. 2, 2009

(54) CURCUMIN-HYALURONAN COMPOUNDS

(75) Inventors: Soumitra Basu, Kent, OH (US); Arijit Das, Kent, OH (US)

Correspondence Address: HAHN LOESER & PARKS, LLP, One GOJO Plaza, Suite 300, AKRON, OH, 44311-1076, US

(73) Assignee: KENT STATE UNIVERSITY, Kent, OH (US)

(21) Appl. No.: 11/966,398

(22) Filed: Dec. 28, 2007

(51) Int. Cl. C12N 5/06; C07H 1/00

(52) U.S. Cl. 435/375; 536/123.1

(57) ABSTRACT

A dissociable complex is disclosed. The dissociable complex includes at least one molecule of curcumin, at least one molecule of hyaluronic acid, and at least one linker molecule, wherein a first portion of the linker molecule is bonded to the curcumin and a second portion of the linker molecule is bonded to the hyaluronic acid.

The invention relates water-soluble, bio-available curcumin-modified compounds and methods. More particularly, the invention relates to modifying curcumin with a biopolymer to form a water-soluble, bio-available compound useful in anti-cancer, antioxidant and anti-inflammatory applications, the treatment of cancer and rheumatoid arthritis cells or other diseased tissue or cells, or other applications.

### BACKGROUND OF THE INVENTION

Curcumin, also known as diferuloymethane, is a natural product obtained from the widely used spice turmeric, and has been in use in traditional medicine for many centuries. It has a wide range of medicinal effects on various diseases, and is currently in clinical trial. A major drawback of curcumin and related products is almost complete insolubility in water, resulting in very poor bioavailability. Often gram level quantities of curcumin are required to elicit a clinical response. The high dosage level required to elicit a pharmacological response, however has been shown to result in elevated levels of toxicity in previous studies.

Hyaluronan, also known as hyaluronic acid, is considered a non-sulfated glycosaminoglycan. It is a linear polysaccharide, with alternating units of D-glucuronic acid and N-acetyl-D-glucosamine having molecular weight of 10⁵-10⁷. Hyaluronic acid is naturally found in many tissues of the body such as skin, cartilage, as well as the vitreous humor.

In the body, hyaluronan is an important component of cartilage, where it is present as a coat around each cell (chondrocyte) in the cartilage. When proteoglycan monomers bind to hyaluronan in the presence of various proteins, large highly negatively charged aggregates form. These aggregates imbibe water and are responsible for the resilience of cartilage, i.e. the resistance to compression. Therefore, hyaluronan is a suitable candidate for biomedical applications targeting these tissues and those throughout the body. Furthermore, it has been shown that ovarian, colon, breast, epithelial, stomach and acute leukemia cancer cell and rheumatoid arthritis cell over-express hyaluronic acid-binding receptors CD44, and these cells exhibit enhanced binding and internalization of hyaluronic acid.

Experiments have been performed to improve the solubility of curcumin. However, these experiments have not been able to overcome the fact that curcumin is covalently bonded to other molecules in such a fashion that free curcumin is not available to act upon the desired target(s). Additionally, the experiments did not target the delivery of curcumin to the diseased tissue or cells, i.e the compounds can potentially be absorbed into any tissue or cells. Efficacy, and non-specific toxicity can be improved if the drug can only be delivered to the site where it is needed.

Notwithstanding the state of the art as described herein, there is a need for further improvements in the preparation of water-soluble, bio-available curcumin-modified compounds that are useful in treating cancer and rheumatoid arthritis cells or for other purposes.

### SUMMARY OF THE INVENTION

In one embodiment of the invention, a dissociable complex is disclosed. The dissociable complex includes at least one molecule of curcumin, at least one molecule of hyaluronic acid, and at least one linker molecule, wherein a first portion of the linker molecule is bonded to the curcumin and a second portion of the linker molecule is bonded to the hyaluronic acid.

In another embodiment of the invention, a method of forming a dissociable complex is disclosed. The method includes the steps of forming at least one linker-functionalized hyaluronic acid molecule, wherein the linker molecule has a first portion bonded to the hyaluronic acid and a second bondable portion, and reacting at least one molecule of curcumin with the at least one linker-functionalized hyaluronic acid molecule, wherein the second bondable portion of the linker molecule bonds to the curcumin.

In yet another embodiment of the invention, a method of treating diseased cells is disclosed. The method includes the steps of forming a dissociable complex, wherein the dissociable complex is formed by creating at least one linker-functionalized hyaluronic acid molecule, wherein the linker molecule has a first portion bonded to the hyaluronic acid and a second bondable portion, and mixing at least one molecule of curcumin with the at least one linker-functionalized hyaluronic acid molecule, wherein the second bondable portion of the linker molecule bonds to the curcumin through formation of an acid cleavable bond, administering the dissociable complex, subjecting the dissociable complex to a pH of less than about 6.5, wherein the pH of less than about 6.5 disassociates the acid cleavable bond between the curcumin and the linker molecule to form at least one unassociated molecule of curcumin, and allowing the at least one unassociated molecule of curcumin to react with the diseased cells. In yet another embodiment of the invention, a pH of less than about 5.5 disassociates the acid cleavable bond between the curcumin and the linker molecule to form at least one unassociated molecule of curcumin, and allowing the at least one unassociated molecule of curcumin to react with the diseased cells.

In yet a further embodiment of the invention, a complex includes at least one molecule of curcumin, at least one molecule of hyaluronic acid, and at least one linker molecule, wherein a first portion of the linker molecule is bonded to the curcumin and a second portion of the linker molecule is bonded to the hyaluronic acid and wherein the linker molecule forms a substantially non-acid labile covalent bond between the curcumin and the linker molecule.

Other aspects of the invention will become apparent upon a reading of the following description in conjunction with the drawings.

### BRIEF DESCRIPTION OF THE DRAWINGS

FIG. 1 is a representation of the dissociable complex showing the acid cleavable bond between the linker molecule and curcumin;

FIG. 2 is a representation of the complex that includes a substantially non-acid labile bond;

FIG. 3 represents confocal microscopy images representing the uptake of curcumin and the dissociable complex into cisplatin resistant ovarian cancer cell line (C13);

FIG. 4 represents confocal microscopy images representing the uptake of curcumin and the dissociable complex into patient-derived rheumatoid arthritis (RA-8001) cells; and

FIG. 5 represents confocal microscopy images representing the uptake of curcumin and the dissociable complex into non-cancerous (NIH3T3) cells.

### BRIEF DESCRIPTION OF THE INVENTION

The potential medicinal uses for curcumin include anti-cancer, antioxidant and anti-inflammatory properties. However, curcumin is known to have a low solubility in water and thus, exhibits a low oral bio-availability. Given the lack of solubility in water and low bio-availability, curcumin is quickly metabolized and excreted in conjunction with bile within the body. To solve the problems of water insolubility and poor bioavailability of curcumin, hyaluronic acid, in conjunction with a linker molecule, is used to chemically modify curcumin thus rendering the resulting dissociable curcumin-hyaluronan complex or bioconjugate compound water-soluble and bio-available, thus rendering the complex with improved pharmacological properties when compared to curcumin alone. The possible routes of administration of the dissociable curcumin-hyaluronan complex include intravenously, intraperitoneally, through localized injection, as a cream, or in an encapsulated form.

In one embodiment of the invention, the dissociable curcumin-hyaluronan complex is formed by first chemically modifying hyaluronic acid through the reaction with a linker molecule to form a linker-functionalized hyaluronic acid molecule. The linker molecule has a first portion bonded to the hyaluronic acid and a second bondable portion capable of bonding with a molecule of curcumin. In one example, the linker molecule is a dihydrazide. In another example, the linker molecule is represented by the formula:

CₙH₂ₙ₊₂N₄O₂, wherein n ranges from about 4 to about 12.

Examples of suitable dihydrazides include adipic dihydrazide, suberic dihydrazide, pimelyl dihydrazide, succinic dihydrazide and 1,12-dodecanedioyl dihydrazide.

Experimentally, the preparation of the linker-functionalized hyaluronic acid molecule is completed in a step-wise manner. First, low molecular weight hyaluronic acid is prepared from the degradation of high molecular weight (about 1.5 MDa) with a pH 6.5 phosphate buffered saline buffer (about 4 mg/mL) in the enzyme hyaluronidase (about 10 U/mg hyaluronic acid). Next, in one example, 1-ethyl-3-(3-dimethylamino)-propyl)carbodiimide (EDC) is reacted with the low molecular weight hyaluronic acid in the presence of a molar excess of the linker molecule. In one embodiment, the linker molecule is adipic dihydrazide. The impure product was purified by dialyzing against water, utilizing a molecular weight cut-off of between about 12,000-14,000) for about 96 hours and then filtered through a 0.2 μm cellulose acetate membrane and lyophilized for about 48 hours.

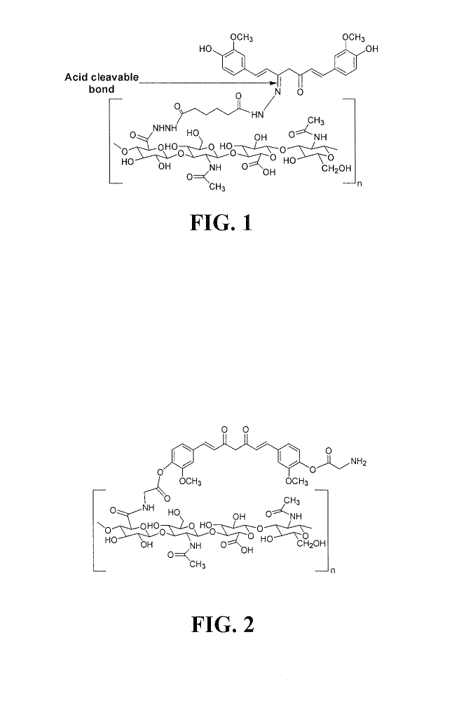

Next, a mixture of linker-functionalized hyaluronic acid molecule and curcumin are stirred in presence of an amount (i.e. one drop) of acetic acid. The reaction was stirred for about 5 days to maximize the reaction between the linker-functionalized hyaluronic acid molecule and the carbonyl group of curcumin. After the completion of the reaction, water was added to precipitate the unreacted curcumin, and the mixture was centrifuged in a 15 ml tube at 4000 rpm for 30 minutes. The supernatant contained dissociable curcumin-hyaluronan complex which was dialyzed (MW cutoff 12000-14000) against water for 96 h. The presence of curcumin in dissociable curcumin-hyaluronan complex was determined by UV absorption spectra at 429 nm and the curcumin loading on hyaluronic acid was calculated from A₄₂₉ value obtained from the spectra. The final dissociable curcumin-hyaluronan complex having a substantially labile, acid cleavable covalent bond is seen in FIG. 1. In this context, the term “substantially labile, acid cleavable covalent bond” is defined as a covalent bond between the linker molecule and curcumin that is susceptible to cleavage at an acidic pH that includes a lysosomal pH of about 5.0.

In order to test the activity of the dissociable curcumin-hyaluronan complex which has a labile, acid cleavable covalent bond, a curcumin-hyaluronan complex having a substantially non-acid labile covalent bond was prepared. In this context, the term “substantially non-acid labile covalent bond” is defined as a covalent bond between the linker molecule and curcumin that is substantially not susceptible to cleavage at an acidic pH that includes a lysosomal pH of about 5.0.

In this study, the complex having the substantially non-acid labile covalent bond is formed by preparing 1,7-Bis(4-O-glycinoyl-3-methoxyphenyl)-1,6-heptadiene-3,5-dione. In this procedure, N-phthaloylglycinoyl chloride (about 2.5 mmol) was added to a stirred solution of curcumin (about 1 mmol) in dry pyridine. Stirring was continued for about 7 hours until completion of the reaction. The reaction mixture was then poured into crushed ice and extracted with ethyl acetate. The ethyl acetate extract was washed with brine and dried with (Na₂SO₄). The crude product was obtained by evaporation of the solvent. The crude product was then treated with ammonia:pyridine (9:1, v/v) at room temperature for about 2 min. The product was then concentrated and purified by column chromatography over silica gel using dichloromethane:methanol gradient to provide a substantially pure product. Next, the 1,7-Bis (4-O-glycinoyl-3-methoxyphenyl)-1,6-heptadiene-3,5-dione was then added to a stirred solution of hyaluronic acid and 1-ethyl-3-(3-(dimethylamino)-propyl)carbodiimide and the stirring was continued for at least 12 hours. The product was purified by dialyzing (with a molecular weight cutoff of about 12000-14000) against water for 96 hours. The presence of curcumin in the curcumin-hyaluronan complex having a substantially non-acid labile covalent bond was determined by UV absorption spectra at 401 nm. The curcumin loading was determined by A₄₀₁ value obtained from UV absorption spectra and it was 1.3%.

Cellular uptake of dissociable curcumin-hyaluronan complex and curcumin was evaluated via in vitro cell culture in the human ovarian cancer cell C-13 (cis-platin resistance), arthritis cell (RA 8001) which overexpresses hyaluronic acid-receptors and the mouse fibroblast NIH3T3 cell as a negative control. In particular, C13 (cisplatin resistance) human ovarian cancer cell and RA 8001, rheumatoid arthritis cells, and NIH3T3 mouse fibroblast cells were plated in an 8-well chamber slide at a concentration of 10,000 cells per well and cultured for 24 hours. The cultured media was replaced by the media containing 2 μM of acid dissociable curcumin-hyaluronan complex or free curcumin. The cells were incubated for 1 hour, 8 hours, 24 hours for C13 cancer cells and NIH3T3 cells and 24 hours, 48 hours for RA 8001 cells. Following incubation the cells were washed with media and DPBS buffer (two times). The cellular uptake was measured by Confocal Fluorescence Microscopy at different time points with dissociable curcumin-hyaluronan complex and free curcumin in human ovarian cancer cell (C-13) and fibroblast-like synoviocytes derived from arthritis patients (RA 8001). The confocal images of the internalized drug complex (dissociable curcumin-hyaluronan complex) and free curcumin were recorded immediately using FITC setting with 488 nm excitation band and 522 nm 32 band pass filter.

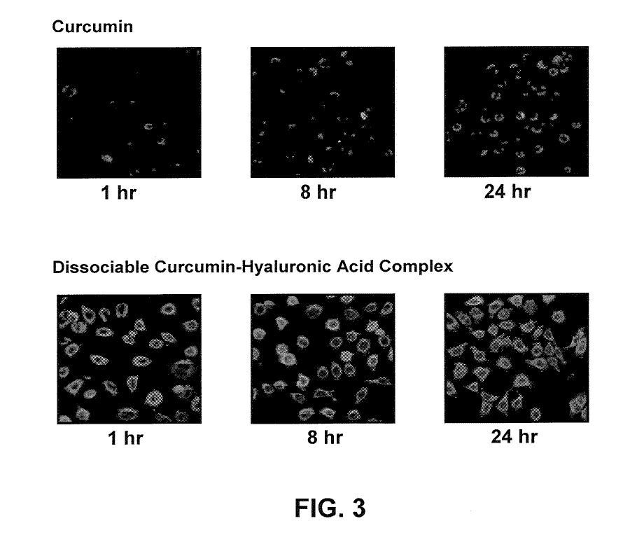

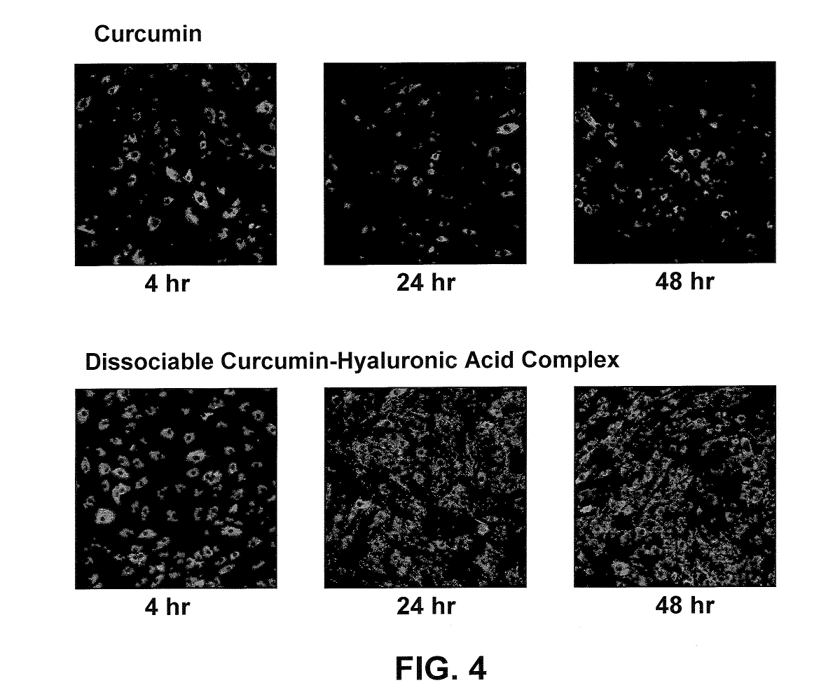

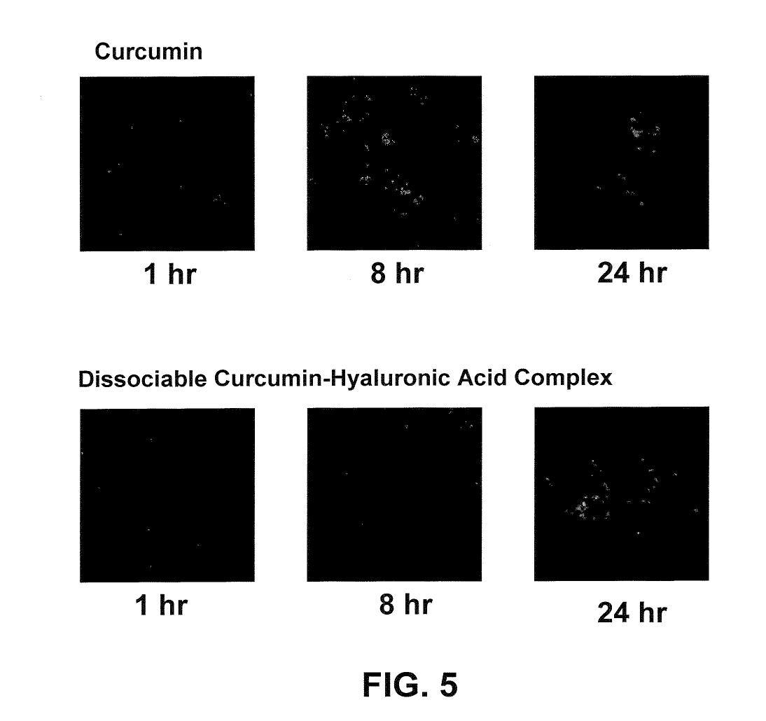

Cytotoxicity effect of dissociable curcumin-hyaluronan complex, the curcumin-hyaluronan complex having a substantially non-acid labile covalent bond and free curcumin was detected by MTS (3-(4,5-dimethylthiazol-2yl)-5-(3-carboxymethoxyphenyl-2-(4-sulfophenyl)-2H-tetrazolium) assay. The formazan color intensity was measured directly from 96 well plates in terms of absorbance at 490 nm and 630 nm using a 96-well multi-scanner (MRX Revelation; Dynex Technologies). For calculating cell viability (%) all the appropriate correction was made and the IC₅₀ values were determined from sigmoidal fitting of the data. FIGS. 3-5 represent confocal microscopy images representing the uptake of curcumin and the dissociable complex into the C-13, NIH3T3 and RA-8001 cell lines.

ImageJ Software (http://rsb.info.nih.gov/ij/) was used to calculate the difference in signals representing the cellular uptake between the dissociable curcumin-hyaluronan complex and curcumin alone in the C-13 and the NIH3T3 cell lines. In particular, the difference in cellular uptake is:

C-13 Cell Line

| | Cellular Uptake |
|---|---|
| | (dissociable curcumin-hyaluronan |
| Time | complex/curcumin) |
| 1 hour | 6.2 fold increase |
| 8 hour | 4.0 fold increase |
| 24 hour | 8.2 fold increase |

NIH3T3 Cell Line

| | Cellular Uptake |
|---|---|
| | (dissociable curcumin-hyaluronan |
| Time | complex/curcumin) |
| 1 hour | 1.0 fold increase |
| 8 hour | 0.9 fold increase |
| 24 hour | 1.1 fold increase |

It was observed that the IC₅₀ value for dissociable curcumin-hyaluronan complex was several fold lower (better) compared to the curcumin-hyaluronan complex having a substantially non-acid labile covalent bond and free curcumin as seen in Table 1.

TABLE 1

| | Hyaluronic | | |
|---|---|---|---|
| | Acid-Curcumin | Hyaluronic Acid-Curcumin | |
| | Dissociable | Non-Dissociable | |
| Cell line | Complex | Complex | Curcumin |
| MDAMB231 | 6.16 ± 1.155 | 10.27 ± 3.1 | 27.49 ± 3.36 |
| C-13 | 3.48 | 14.37 | 22.25 |
| MCF7 | 6.34 | nd | 15^{a} |
| MCF7 (Dox | 7.73 ± .465 | nd | 27.45 ± .892 |
| resistance) | | | |
| NIH3T3 | 12.51 ± 3.16 | | 23.85 ± 3.19 |
| RA-8001 | 3.69 ± .38 | 6.91 ± .198 | 11.08 ± 2.3 |

nd = not determined
^{a}= Simon et al., Cancer Lett., (1998), 129, 111

In the dissociable curcumin-hyaluronan complex, given the presence of an acid labile and cleavable hydrazone bond between the curcumin and the linker molecule, once the acid cleavable hydrazone bond is hydrolyzed at low pH, for example a pH of about 5.5 as found in the lysosome of a cell, curcumin disassociates from the curcumin-hyaluronan complex and becomes available inside the cell to produce its pharmacological response. This is clearly shown by the IC₅₀ difference between the acid labile dissociable curcumin-hyaluronan complex and the curcumin-hyaluronan complex having a substantially non-acid labile covalent bond as detailed in Table 1.

In general, it has been shown that the dissociable curcumin-hyaluronan complex is capable of uptake in various types of cancer cells and rheumatoid arthritis cells since it can bind to surface proteins that are found on the surface of these diseased cells in larger numbers compared to normal tissue. Attachment of the hyaluronan moiety to curcumin led to improved water solubility, cellular bioavailability and efficacy as evaluated in cell culture models. The dissociable curcumin-hyaluronan complex is effective in killing cancer cells and patient derived rheumatoid arthritis cells. The possible routes of administration of the dissociable curcumin-hyaluronan complex can be intravenous, intraperitoneal, localized injection into joints, as a cream, and in an encapsulated form. As an alternative example, for effective delivery of curcumin, it can be attached to hyaluronic acid through gold nano particles. Gold or other nano particles may be able to carry large numbers of drug molecules per particle. In an example, a thiol functionalized curcumin may be reacted with a gold nano particle. In order to target a curcumin coated gold nano particle, a targeting ligand, such as a hyaluronic acid, antibody or other small molecule ligand may be attached to the gold nano particle. Further, polyethylene glycol or other water soluble polymers may be attached to curcumin, which may increase the solubility and bioavailability of the curcumin. Alternatively, attaching with folic acid, monoclonal antibody through different hydrophilic polymers (ie, HPMA) with curcumin can also increase one of more of the properties of solubility, bioavailability and/or targeted delivery.

Based upon the foregoing disclosure, it should now be apparent that the use of water-soluble, bio-available curcumin-modified compounds useful in anti-cancer, antioxidant and anti-inflammatory applications, such as in the treatment of cancer and rheumatoid arthritis cells, as described herein, will carry out the objects set forth hereinabove. It is, therefore, to be understood that any variations evident fall within the scope of the claimed invention and thus, the selection of specific component elements can be determined without departing from the spirit of the invention herein disclosed and described.

What is claimed is:

1. A dissociable complex comprising:
at least one molecule of curcumin;
at least one molecule of hyaluronic acid; and
at least one linker molecule, wherein a first portion of the linker molecule is bonded to the curcumin and a second portion of the linker molecule is bonded to the hyaluronic acid.

2. The complex of claim 1, wherein the at least one linker molecule is a dihydrazide.

3. The complex of claim 2, wherein the dihydrazide is represented by the formula:
CₙH₂ₙ₊₂N₄O₂
wherein n ranges from about 4 to about 12.

4. The complex of claim 3, wherein the dihydrazide is adipic dihydrazide.

5. The complex of claim 1, wherein the complex is water-soluble.

6. The complex of claim 1, wherein the first portion of the linker molecule bonded to the curcumin is an acid cleavable bond.

7. The complex of claim 6, wherein a pH of less than about 6.5 hydrolyzes the acid cleavable bond between the curcumin and the linker molecule to form an unassociated molecule of curcumin.

8. The complex of claim 7, wherein a pH of less than about 5.5 hydrolyzes the acid cleavable bond between the curcumin and the linker molecule to form an unassociated molecule of curcumin.

9. A method of forming a dissociable complex, the method comprising the steps of:
forming at least one linker-functionalized hyaluronic acid molecule, wherein the linker molecule has a first portion bonded to the hyaluronic acid and a second bondable portion; and
reacting at least one molecule of curcumin with the at least one linker-functionalized hyaluronic acid molecule, wherein the second bondable portion of the linker molecule bonds to the curcumin.

10. The method of claim 9, wherein the at least one linker molecule is a dihydrazide.

11. The complex of claim 10, wherein the dihydrazide is represented by the formula:
CₙH₂ₙ₊₂N₄O₂
wherein n ranges from about 4 to about 12.

12. The method of claim 11, wherein the dihydrazide is adipic dihydrazide.

13. The method of claim 9, wherein the complex is water-soluble.

14. The method of claim 9, wherein the second portion of the linker molecule bonded to the curcumin is an acid cleavable bond.

15. The complex of claim 14, wherein a pH of less than about 6.5 hydrolyzes the acid cleavable bond between the curcumin and the linker molecule to form an unassociated molecule of curcumin.

16. The complex of claim 15, wherein a pH of less than about 5.5 hydrolyzes the acid cleavable bond between the curcumin and the linker molecule to form an unassociated molecule of curcumin.

17. A method of treating diseased cells, the method comprising the steps of:
forming a dissociable complex, wherein the dissociable complex is formed by creating at least one linker-functionalized hyaluronic acid molecule, wherein the linker molecule has a first portion bonded to the hyaluronic acid and a second bondable portion, and mixing at least one molecule of curcumin with the at least one linker-functionalized hyaluronic acid molecule, wherein the second bondable portion of the linker molecule bonds to the curcumin through formation of an acid cleavable bond;
administering the dissociable complex;
subjecting the dissociable complex to a pH of less than about 6.5, wherein the pH of less than about 6.5 hydrolyzes the acid cleavable bond between the curcumin and the linker molecule to form at least one unassociated molecule of curcumin; and
allowing the at least one unassociated molecule of curcumin to react with the diseased cells.

18. The method of claim 17, wherein the at least one linker molecule is a dihydrazide.

19. The complex of claim 18, wherein the dihydrazide is represented by the formula:
CₙH₂ₙ₊₂N₄O₂
wherein n ranges from about 4 to about 12.

20. The method of claim 19, wherein the dihydrazide is adipic dihydrazide.

21. The method of claim 17, wherein the complex is water-soluble.

22. The method of claim 17, wherein the dissociable complex is administered intravenously, intraperitoneally, through localized injection, as a cream or through ingestion of an encapsulated form of the dissociable complex.

23. The method of claim 17, wherein a pH of less than about 5.5 hydrolyzes the acid cleavable bond between the curcumin and the linker molecule to form an unassociated molecule of curcumin.

24. A complex comprising:
at least one molecule of curcumin;
at least one molecule of hyaluronic acid; and
at least one linker molecule, wherein a first portion of the linker molecule is bonded to the curcumin and a second portion of the linker molecule is bonded to the hyaluronic acid and wherein the linker molecule forms a substantially non-acid labile covalent bond between the curcumin and the hyaluronic acid.
